(19) United States
(12) Patent Application Publication
(10) Pub. No.: US 2009/0192408 A1
(43) Pub. Date: Jul. 30, 2009

(54) SURGICAL SITE MARKER DELIVERY SYSTEM

(76) Inventor: Joseph L. Mark, Indianapolis, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/020,921

(22) Filed: Jan. 28, 2008

(51) Int. Cl. A61B 10/00

(52) U.S. Cl. 600/562

(57) ABSTRACT

A marker delivery system is provided that includes a delivery tube and a push rod. The delivery tube is defined by distal and proximal ends and includes a distal opening that communicates with a lumen extending through the delivery tube and a curved distal end portion. The distal opening is positioned distally of the curved distal end portion. The push rod is configured for slidable engagement within the lumen of the delivery tube and is sized to extend to at least the distal opening of the delivery tube when the push rod is fully inserted within the delivery tube to push out one or more site markers disposed within the lumen of the delivery tube.

### TECHNICAL FIELD

The present disclosure generally relates to a biopsy site marker delivery system that is used with a biopsy tissue cutting device.

### BACKGROUND

In the field of breast cancer, stereotactically guided and percutaneous biopsy procedures have increased in frequency as well as in accuracy as modern imaging techniques allow the physician to locate lesions with ever increasing precision. However, for a given biopsy procedure, a subsequent examination of the biopsy site may be very often desirable.

For example, in those cases where the lesion may be found to be benign, a follow-up examination of the biopsy site may be conducted at a later time. Where the lesion may be found to be malignant, the physician may want to place additional site markers to help guide the surgeon to the malignancy.

A number of procedures and devices for marking and locating particular tissue locations are known in the prior art. For example, location wire guides are well known for locating lesions, particularly in the breast. One such known device includes a tubular introducer needle and an attached wire guide, which has at its distal end, a helical coil configuration for locking into position about the targeted lesion. The needle is introduced into the breast and guided to the lesion site using an imaging system of a known type, for example, X-Ray, ultrasound or magnetic resonance imaging (MRI), at which time the helical coil at the distant end may be deployed about the lesion. Then, the needle is removed from the wire guide, which remains locked in position distally about the lesion for guiding a surgeon down the wire to the lesion site during subsequent surgery. While such a location system may be effective, it is obviously intended and designed to be only temporary, and is removed once the surgery or other procedure has been completed.

It is also known to employ biocompatible dyes or stains to mark breast lesions. First, a syringe containing the colorant is guided to the detection lesion, using an imaging system. Later, during the extraction procedure, the surgeon harvests a tissue sample from the stained tissue. However, while such staining techniques can be effective, it may be difficult to precisely localize the stain. Also, the stains are difficult to detect fluoroscopically and may not always be permanent.

Additionally, it is known to implant markers directly into a patient's body using an invasive surgical technique. This enables a practitioner to later return to the site of the graft by identifying the rings, for evaluation purposes.

Each of the above systems and methods for marking a biopsy site has disadvantages associated with effectiveness, accuracy, and invasive surgical techniques. Accordingly, a site marker delivery system for delivering a marker to a biopsy site, and deploying the marker at the site effectively, accurately, and without the need for additional invasive surgical procedures is needed.

### SUMMARY

A marker delivery system is provided that includes a delivery tube and a push rod. The delivery tube is defined by distal and proximal ends and includes a distal opening that communicates with a lumen extending through the delivery tube and a curved distal end portion. The distal opening is positioned distally of the curved distal end portion. The push rod is configured for slidable engagement within the lumen of the delivery tube and is sized to extend to at least the distal opening of the delivery tube when the push rod is fully inserted within the delivery tube to push out one or more site markers disposed within the lumen of the delivery tube.

### BRIEF DESCRIPTION OF THE DRAWINGS

FIG. 1 is an elevational perspective view of a site marker delivery system, having an elongated delivery tube with a pre-curved end.

FIG. 1A is an enlarged, perspective view of the pre-curved end of the delivery tube of FIG. 1.

FIG. 2 is a partial cross section of a portion of the site marker delivery system in FIGS. 1 and 1A, prior to ejection of a site marker.

FIG. 3 is a partial cross section of a portion of the site marker delivery system in FIG. 2, ejecting the site marker.

FIG. 4 is a partial cross section of a portion of the site marker delivery system in FIG. 2, ejecting the site marker, and illustrating a push rod protruding a predetermined distance D from the elongated delivery tube.

FIG. 5 is an alternative illustration of a partial cross section of a portion of a site maker delivery system including a push rod disposed within the delivery tube.

FIG. 6 is an elevational perspective view of an alternative elongated delivery tube.

FIG. 7 is an elevational, partially sectioned perspective view of the elongated delivery tube of FIG. 6, disposed within a tissue cutting device.

FIG. 8 is a front elevational view of the delivery tube of FIG. 6.

FIG. 9 is a partial cross section of a portion of an alternative site marker delivery system including a push rod disposed within the delivery tube.

FIG. 10 is a perspective view of a tissue cutting device.

FIG. 11 is a side elevational view of the tissue cutting device.

FIG. 12 is a side elevational view of the tissue cutting device with a site marker delivery system being engaged therewith.

FIG. 13 is a side elevational view of the tissue cutting device with a site marker delivery system fully engaged therewith.

### DETAILED DESCRIPTION

A site marker delivery system 300 that is used in combination with a tissue cutting device TD for efficiently, accurately and sanitarily depositing a marker at a biopsy site, is disclosed herein. The site marker delivery system 300 is at least partially formed from medical grade materials, for example, stainless steel, titanium alloy, plastic and rubber. The site marker delivery system 300 may also include universal features that permits the system to be used with a number of different tissue cutting devices of different sizes, particularly with regard to an inner diameter of an inner cannula and the length of the cutting element of a particular tissue cutting device. The system 300 may be adaptable for use in conventional biopsy procedures which may be performed manually and/or stereo-tactically.

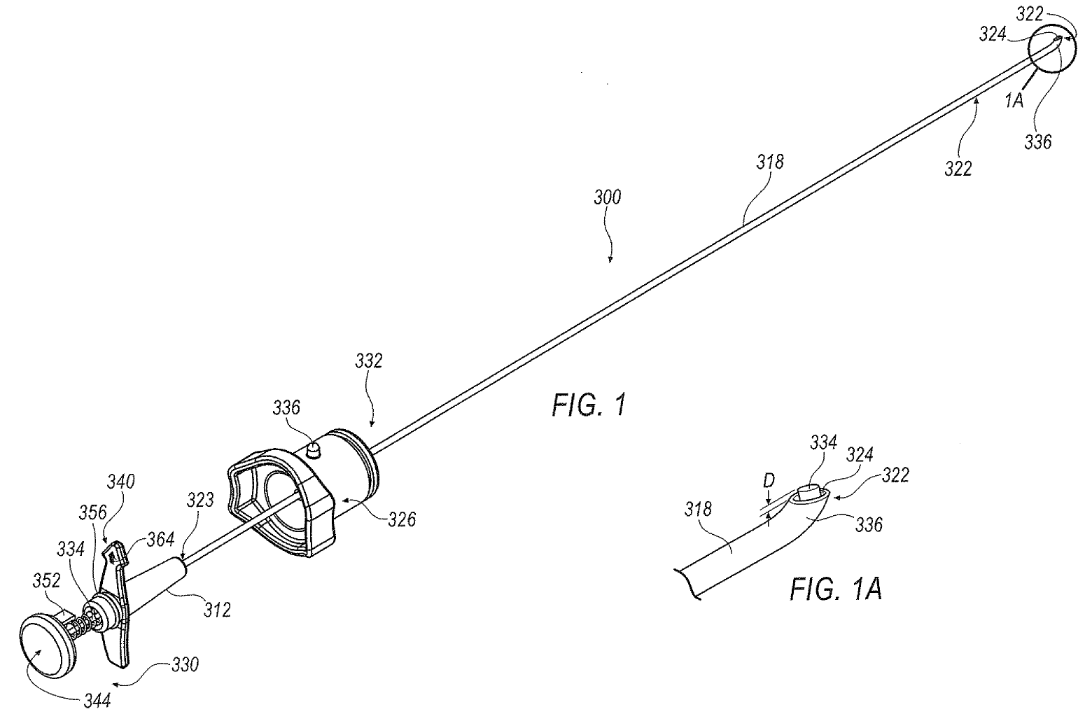

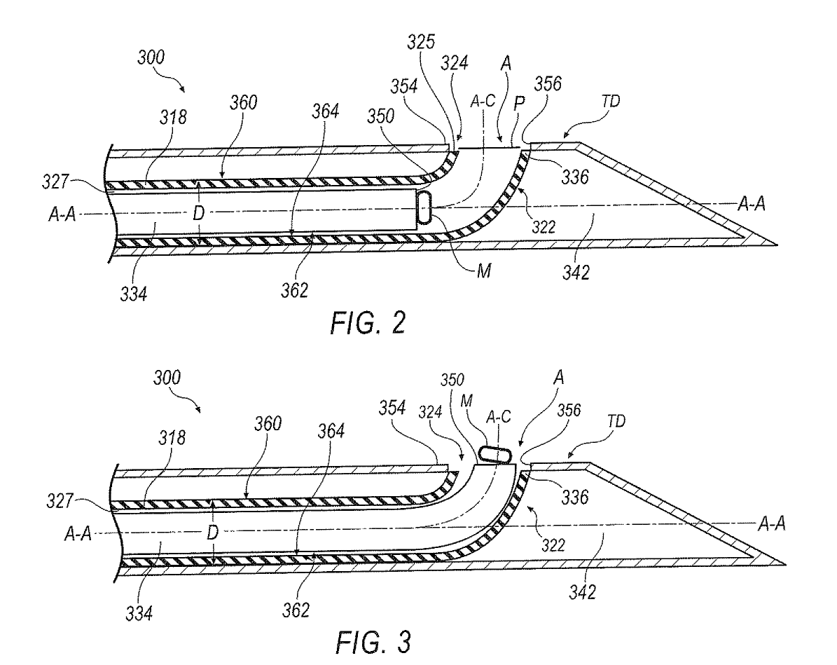

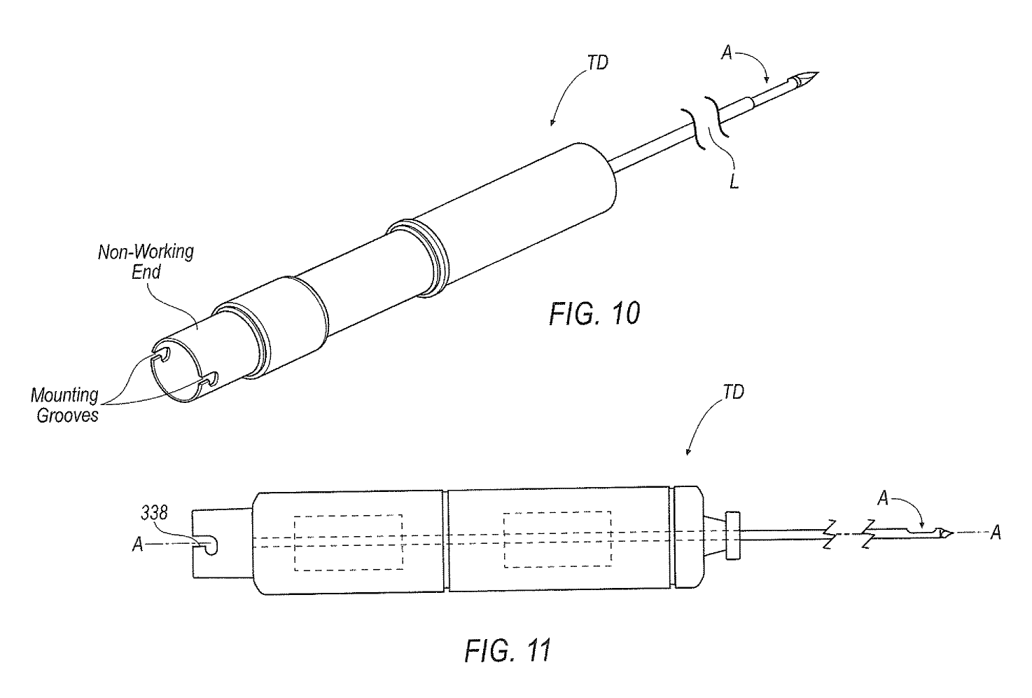

Referring to FIG. 1, an exemplary illustration of a site marker delivery system 300 is shown. Site marker delivery system 300 includes an elongated delivery tube 318 and a push rod 334, as shown in FIG. 10. Elongated delivery tube 318 is defined by a distal end 322 and a proximal end 323. As may be seen, distal end 322 is formed so as to curve upwardly. A distal opening 324 is formed at a distal end 322 (as best seen in FIG. 1A). The distal opening 324 opens into and is in communication with a lumen 327 (best seen in FIG. 2).

Delivery tube 18 may be connected to a hub 312 that is similar to that disclosed in commonly owned and co-pending U.S. patent application Ser. No. 11/238,295, the contents of which are incorporated herein by reference in its entirety. Hub 312 includes a channel (not seen) that may receive the proximal end of 323 of delivery tube 18. Alternatively, the delivery tube 18 may be fixedly secured to an opening of the channel.

Hub 312 may further include an indicator 340 positioned on end of hub 312. The indicator 340 is indicative of the circumferential orientation of the distal opening 324 of the delivery tube 318, as the orientation is difficult to determine when the delivery tube 318 is positioned within a patient. The indicator 340 may include a marking 364 that allows the indicator 340 to be visible in low light conditions.

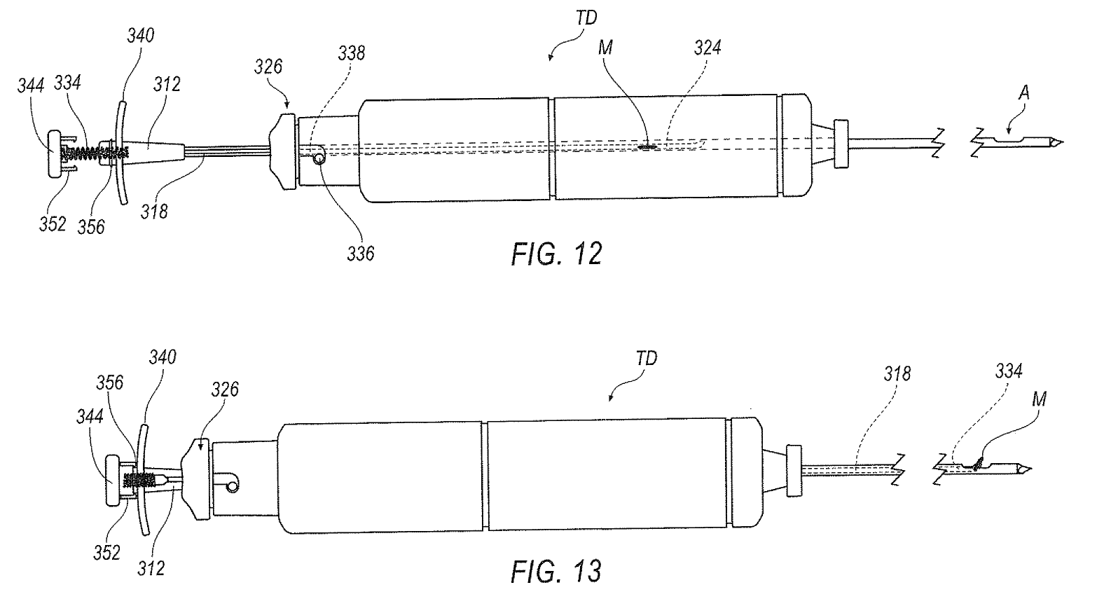

The site marker delivery system may further include a tube guide 326, such as that which is disclosed in commonly owned and co-pending U.S. patent application Ser. No. 11/238,295. Tube guide is configured to be selectively and detachably connected to a non-working end of a tissue cutting device TD (see, e.g., FIGS. 10-13). The tube guide 326 is formed with a channel therethrough (not shown), and includes at least one attachment mechanism. In one embodiment, the attachment mechanism is an attachment pin 336 disposed on an exterior wall of the tube guide 326. The attachment pin 336 is constructed for engaging a complimentary mounting groove 338 formed on the non-working end of the tissue cutting device TD so as to key the tube guide 326 to the tissue cutting device (See FIGS. 12-13).

Push rod 334 is sized so as to fit in sliding engagement within delivery tube 318 and towards distal end 322, as explained in further detail below. The push rod 334 is operable to advance a site marker M seated in lumen 327 out of distal opening 324. As such, the push rod 334 includes an outer diameter that is sized to at least partially close or block distal opening 324 such that after the site marker M has been deployed, it cannot fall back through aperture 324 and return to the lumen 327. In one embodiment, as best seen in FIG. 1A, the push rod 334 has a predefined length such that at least a portion of the push rod 334 protrudes from the distal opening 324 of the tube 318 by a predetermined distance D when site marker M is discharged from the distal opening 324.

The push rod 334 of the site marker delivery system 300 may further include a deployment trigger 344, such as that which is disclosed in commonly owned and co-pending U.S. patent application Ser. No. 11/238,295. The deployment trigger 334 may be formed as a push plate. In one embodiment, the deployment trigger 334 may further include at least one retaining arm 352 that operates to engage a complimentary retaining rim 356 formed on the hub 312. The retaining arm 352 and retaining rim 356 operate to lock to the push rod 334 within the lumen 327 of the delivery tube 318 after the site marker M has been advanced through the distal opening 324. The retaining arm 352/retaining rim 356 configuration may also provide a tactile and audible indication to a user that the site marker M has been deployed.

Referring to FIGS. 1A-4, distal end 322 of the delivery tube 318 includes a pre-curved portion 336. The pre-curved portion 336 is configured so that the distal opening 324 extends generally radially towards an aperture of a tissue cutting device TD, when the delivery tube 318 is inserted into the tissue cutting device TD. In one embodiment, the distal opening 324 is configured to be generally parallel to at least a portion of an aperture formed in a side-wall of a tissue cutting device TD, as discussed in greater detail below. Moreover, the pre-curved portion 336 allows for the delivery tube 318, and more specifically the distal opening 324, to be substantially aligned with the aperture of the tissue cutting device TD, prior to the deployment of a site marker M.

The delivery tube 318 is at least partially constructed from a material that is rigid enough to allow the delivery tube 318 to be inserted into a tube guide, such as the tube guide 326, in a straightened condition, while still being able to spring back to the pre-curved position as soon as the tube 318 enters a proximal end of a tube guide 326 (discussed below). It is understood that any material that accommodates this need such as, but not limited to, a high density polyethylene, a urethane, a nylon or a Pebax® may be used. In one embodiment, the tube 318 is at least partially constructed from a MRI compatible material.

As illustrated in FIG. 2, the push rod 334 is selectively operable to push or guide one or more site markers M positioned in lumen 327 of the delivery tube 318 and through an aperture A of a tissue cutting device TD. FIG. 2 illustrates the push rod 334 being slideably passed though the lumen 327 of delivery tube 318, and guiding the site marker M position within the lumen 327. In one embodiment, the elongated delivery tube 318 may selectively interposed within an inner lumen 342 of the tissue cutting device TD. Alternatively, the elongated delivery tube 318 may be disposed within an introducer lumen, such as that disclosed in commonly owned U.S. patent application Ser. No. 10/649,068, the contents of which are incorporated by reference in its entirety.

In the embodiment illustrated in FIGS. 2-4, the site marker delivery system 300 is illustrated as being used with the tissue cutting device TD. As may be seen, aperture A is formed in a portion of a side-wall of the tissue cutting device TD. In the embodiment as illustrated in FIG. 2, the site marker delivery system 300 is configured for side deployment. That is, an axis A-A of the tissue cutting device TD does not extend through aperture A of the tissue cutting device TD. At least a portion of an outer surface 325 of the distal opening 324 is generally parallel to a horizontal plane P that at least partially defines the aperture A.

As discussed above, the push rod 334 at least partially closes or blocks the aperture A when in a deployed configuration (see FIG. 3). That is, after a site marker M has been deployed, the push rod 334 reduces the possibility of the site marker M falling back into either the aperture A of the tissue cutting device TD or the site marker delivery system 300 as either device is removed from a patient. Such an occurrence of the site marker M falling back into either aperture A or distal opening 324 is known as drag-out. Drag-out typically occurs when a marker M is trapped or caught on a side surface 356 of the aperture A of the tissue cutting device TD when the tissue cutting device TD is removed from the biopsy site such that the site marker M is “dragged” out of the biopsy site and may be disposed somewhere in the instrument pathway leading to the biopsy site as the devices are removed from the site. Thus, drag-out may result in the biopsy site not being properly identified, as the marker M will be removed from the biopsy site when the tissue cutting device TD or the site marker delivery device 300 is removed.

As illustrated by FIG. 3, at least a portion of a distal end 350 of the push rod 334 is generally flush with an outer surface 354 of the aperture A of the tissue cutting device TD when the site marker M is deployed. That is, the distal end 350 of the push rod 334 is generally oriented so as to be substantially horizontal such that the distal end 350 is substantially flush in relation to the outer surface 354 when the push rod is in a deployment configuration.

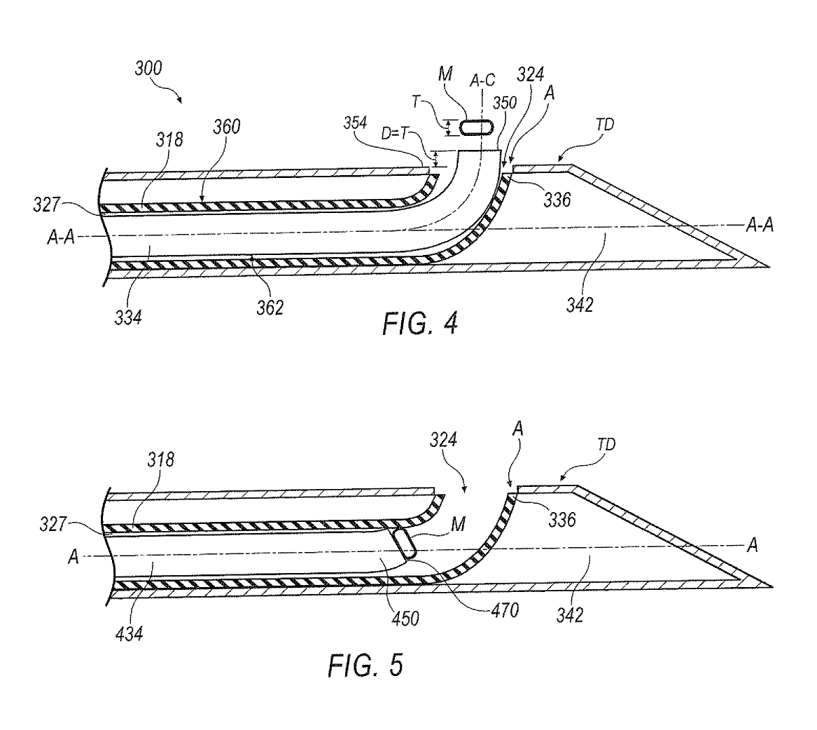

Alternatively, as best seen in FIG. 4, the push rod 334 may be configured so as to extend outwardly from the distal opening 324, as well as the aperture A of the tissue cutting device TD. In one embodiment, the distal end 350 of the push rod 334 protrudes from the aperture A a predetermined distance D that is at least equal to the minimum thickness T of the site marker M that is discharged from the aperture A. By configuring the push rod 334 to advance from the distal opening 324 and/or aperture A, the marker M may be placed further away from both the site marker delivery device 300 and the tissue cutting device and deposited into the biopsy site, thereby reducing the occurrence of drag-out.

The distal opening 324 of site marker delivery device 300 is selectively in communication with the aperture A of the tissue cutting device TD. The push rod 334 is dimensioned to slideably pass through the lumen 327 of delivery tube 318 and towards the aperture A of the tissue cutting device TD. At least a portion of the lumen 327 of tube 318 is defined by a curved axis, AC, as seen in FIG. 4. That is, the pre-curved portion 336, which includes the distal opening 324, is curved radially towards the aperture A of the tissue cutting device TD, when the site marker delivery device 300 is disposed therewithin. The curved axis AC intersects the distal opening 324. Thus, the pre-curved portion 336 allows for the distal opening 324, and in particular the outer surface 325, to be generally parallel to at least a portion of the aperture A formed in a side-wall of the tissue cutting device TD.

The push rod 334 is slidably passed through the lumen of the tube 318 towards the distal end 322, as shown in FIG. 3. The curved axis AC serves to selectively define at least a portion of the push rod 334. More specifically, the curved portion of axis AC of the tube 318 urges the push rod 334, the distal end at least being somewhat flexible, into a curved configuration. That is, as the push rod 334 is guided through the lumen 340 of the delivery tube 318, at least a portion of an inner surface 364 of delivery tube 318 at the distal end 322 is curved, thereby urging the distal end 350 of the push rod 334 towards the distal opening 324 and aperture A. The distal end 350 of the push rod 334 is constructed from a material that will allow for the push rod 334 to be encouraged into a curved configuration, much like the pre-curved end 336 of the tube 318.

In one illustrative embodiment, as seen in FIG. 3, at least a portion of an outer surface 360 of the delivery tube 318 is in contact with an inner surface of the tissue cutting device TD. More specifically, the delivery tube 318 may have an outer diameter D that is substantially contiguous with a lumen 342 of the tissue cutting device TD. Additionally, at least a portion of an outer surface 362 of the push rod 334 may be in sliding contact with the inner surface 364 of the delivery tube 318.

In operation, the delivery tube 318 may be positioned within the tissue cutting device TD for deployment of the site marker M, as follows. First, the distal end 322 of the delivery tube 318 is inserted into the lumen 342 of the tissue cutting device TD. The distal opening 324 is oriented so as to be generally aligned with the aperture A of the tissue cutting device TD, as seen in FIG. 2. For those embodiments that include an indicator 340 (shown in FIG. 1), the user may radially align the marking 364 of the indicator 340 with an indicator on the tissue cutting device (not shown) to ensure that the distal opening 324 is aligned with the aperture A. If a tube guide 326 is provided, the tube guide 326 may be secured to a portion of the tissue cutting device TD to prevent inadvertent dislodgement of the delivery tube 318.

Once the delivery tube 318 is seated within the lumen 342 of the tissue cutting device TD, the push rod 334 is inserted within the lumen 327 of the delivery tube 318. This is accomplished by inserting the distal end 350 of the push rod 334 into the lumen 327 of the delivery tube 318, which contains one or more site markers M therein. The push rod 334 is then advanced through the lumen 327 toward the distal opening 327.

Use of the site marker delivery system 300 will now be described. A method of marking a biopsy site in combination with the tissue cutting device TD is disclosed. The push rod 334 is guided through the lumen 327 of delivery tube 318 by sliding the push rod 334 towards the distal end of the tissue cutting device TD, as seen in FIG. 2. Thus, the push rod 334 is selectively interposed within the lumen 327 of the delivery tube 318, and a portion of the lumen 327 is defined by the curved axis, AC. That is, at least a portion of the lumen 327 of the delivery tube 318 at least partially guides the outer surface 362 of the push rod 334. Thus, as seen in FIG. 3, the push rod 334 will deflect radially towards the distal opening 324 and aperture A of the tissue cutting device TD.

As seen in FIGS. 3 and 4, the site marker M is deposited in the biopsy site by urging the site marker M positioned in the lumen 327 of the delivery tube 318 through the distal opening 324 of the delivery tube 318. More specifically, the site marker M is urged through the aperture A of the tissue cutting device TD. As illustrated in FIG. 4, in one embodiment, at least a portion of the push rod 334 protrudes from the aperture A of the tissue cutting device TD at the predetermined distance D to “kick out” the site marker M, thereby preventing “drag out.”

In another illustration as seen in FIG. 5, least a portion of a distal end 450 of a push rod 434 includes a pre-curved end 470. The pre-curved end 470 extends generally radially to the tissue cutting device TD axis A-A, and is curved towards the distal opening 324 of the tube delivery 318. More specifically, the pre-curved end 470 extends generally radially towards the aperture A of the tissue cutting device TD when the site marker delivery device 300 is inserted therein.

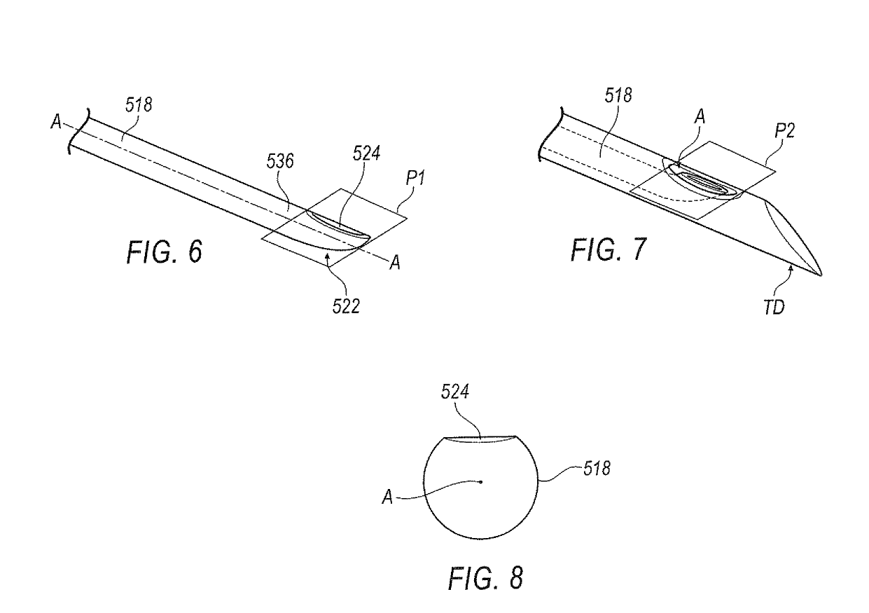

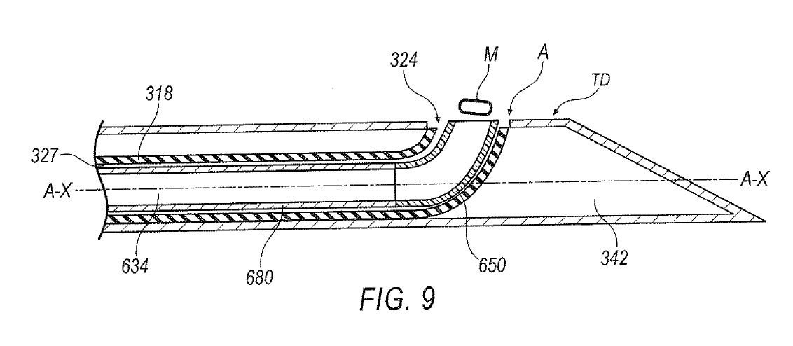

FIG. 6 illustrates an elongated delivery tube 518 that includes a pre-bent end 536, rather than a pre-curved end. That is, the distal opening 524 is formed in a side wall of the delivery tube 518, instead of at a curved distal end of the delivery tube 518. A portion of the delivery tube 518 is sloped adjacent to the distal opening 524 to form the pre-bent end 536.

Indeed, the pre-bent end 536 is formed such that the distal opening 524 extends generally radially towards the aperture A of the tissue cutting device TD, as seen in FIG. 7. That is, a plane P1 that at least partially defines the distal opening 524 (seen in FIG. 6) is generally parallel to a portion of a plane P2 (seen in FIG. 7) that at least partially defines the aperture A. Moreover, as seen in FIG. 8, when the distal end 522 is viewed from the axis A of the delivery tube 518, it should be noted that the profile of the tube 518 is generally planar at the distal opening. Indeed, the axis A of the delivery tube 518 is generally linear. The profile will facilitate entry of the tube 518 into a tube guide, such as the tube guide 26, without the need for the tube 518 to spring back to a pre-curved configuration, when reaching a tissue aperture A. Thus, the tube 518 may be constructed of relatively rigid materials that do not spring back into a pre-curved position. It is understood that any material that accommodates this need such as, but not limited to, a stainless steel or a titanium alloy may be used.

In one illustration of a push rod 634, as seen in FIG. 14, the push rod 634 may be constructed from two different materials. That is, only a distal end 650 of the push rod 634 is constructed of a material that will allow for the distal end 650 to bend or flex within the lumen 340 of the delivery tube 318. However, the remainder of the push rod 634, a main body 680, is constructed from another material that may be stiffer and less flexible than the distal end 650 of the push rod 634. It should be noted that any material that accommodates this need may be used for the main body 680, such as, but not limited to, a stainless steel and a titanium alloy.

All of the push rods 334, 434, and 634 are configured such that the site marker M is unable to migrate towards a proximal end of any of the elongated delivery tubes. In one embodiment, the push rods are constructed from a material that is more flexible and less rigid than the material used to construct any of the tubes 318, 418 or 518. Moreover, the push rods are at least partially constructed from a material that is rigid enough to guide marker M through the lumen 340 of any of the tubes 318, 418 or 518, while still being able to bend within the lumen 340. It is understood that any material that accommodates this need such as, but not limited to, a high density polyethylene, a nylon, a urathane or a Pebax® may be used. Any of the push rods may be at least partially constructed from a MRI compatible material.

While the present disclosure has been particularly shown and described with reference to the foregoing preferred embodiments, it should be understood by those skilled in the art that various alternatives to the embodiments of the disclosure described herein may be employed in practicing the disclosure without departing from the spirit and scope of the disclosure as defined in the following claims. It is intended that the following claims define the scope of the disclosure embodiments within the scope of these claims and their equivalents be covered thereby. This description of the disclosure should be understood to include all novel and non-obvious combinations of elements described herein, and claims may be presented in this or a later application to any novel and non-obvious combination of these elements. The foregoing embodiment is illustrative, and no single feature or element is essential to all possible combinations that may be claimed in this or a later application.

What is claimed is:

1. A site marker delivery system, comprising:
a delivery tube defined by distal and proximal ends; the delivery tube including a distal opening formed at the distal end that communicates with a lumen extending through the delivery tubes and wherein the distal end further includes a curved distal end portion, wherein the distal opening is positioned distally of the curved distal end portion; and
a push rod configured for slidable engagement within the lumen of the delivery tube and sized to extend to at least the distal opening of the delivery tube when the push rod is fully inserted within the delivery tube to push a site marker positioned in the lumen out of the distal opening.

2. The marker deployment device of claim 1, wherein at least a portion of the delivery tube is defined by a generally linear axis.

3. The marker deployment device of claim 1, wherein at least a portion of a distal end of the push rod is curved.

4. The marker deployment device of claim 3, wherein at least a portion of the push rod protrudes from the distal opening by a predetermined distance when the push rod is fully inserted within the delivery tube.

5. The marker deployment device of claim 1, wherein at least a portion of the push rod is constructed from a flexible material.

6. The marker deployment device of claim 5, wherein the push rod is constructed from a first material and a second material, a distal end of the push rod being constructed from the first material and a proximate end of the push rod constructed from the second material, wherein the first material is flexible.

7. The marker deployment device of claim 6, wherein the first material is one of a high density polyethylene, a nylon, a Pebax or a urethane, and the second material is one of a titanium alloy or a stainless steel.

8. The marker deployment device of claim 1, wherein the distal opening is oriented so as to lie in a plane that is substantially parallel to an axis extending through a non-curved portion of the delivery tube.

9. A biopsy site marker delivery system, comprising:
an outer cannula defined by a proximal end and a distal end, wherein the outer cannula defines a lumen and includes an aperture formed in a side-wall of the outer cannula such that the aperture is in communication with the lumen;
a delivery tube defined by distal and proximal ends; the delivery tube including a distal opening formed at the distal end that communicates with a lumen extending through the delivery tube, and wherein the distal end further includes a curved distal end portion, wherein the distal opening is positioned distally of the curved distal end portion, wherein the delivery tube is configured for slidable engagement within the lumen of the outer cannula such that the distal opening of the delivery tube is positioned in communication with the aperture of the outer cannula when the delivery tube is fully inserted within the outer cannula; and
a push rod configured for slidable engagement within the lumen of the delivery tube and sized to extend to at least the distal opening of the delivery tube when the push rod is fully inserted within the delivery tube to push a site marker positioned in the lumen out of the distal opening and through the aperture.

10. The marker deployment device of claim 9, wherein an outer surface of the distal opening is generally parallel to a plane extending across the aperture.

11. The marker deployment device of claim 9, wherein at least a portion of the push rod protrudes from the distal opening by a predetermined distance when the push rod is fully inserted within the delivery tube.

12. The marker deployment device of claim 9, wherein at least a portion of a distal end of the push rod is curved such that the distal end of the push rod extends generally radially towards the aperture when inserted within the delivery tube.

13. The marker deployment device of claim 9, wherein the push rod is constructed from a first material and a second material, a distal end of the push rod constructed from the first material and a proximate end of the push rod constructed from the second material.

14. The marker deployment device of claim 133, wherein the first material is one of a high density polyethylene, a nylon, a Pebax or a urethane, and the second material is one of a titanium alloy or a stainless steel.

15. A method of marking a biopsy site, comprising:
providing a delivery tube that includes a distal opening formed at a distal end thereof that communicates with a lumen extending through the delivery tube, and wherein the distal end further includes a curved distal end portion, wherein the distal opening is positioned distally of the curved distal end portion;
positioning at least one site marker within the lumen of the delivery tube;
providing an outer cannula that defines a lumen and includes an aperture formed in a side-wall of the outer cannula such that the aperture is in communication with the lumen;
interposing the delivery tube within the outer cannula and aligning the distal opening of the delivery tube with the aperture of the outer cannula, the distal opening of the inner tube extending generally radially towards and in selective communication with the aperture;
sliding a push rod at least partially through the lumen of the delivery tube towards the distal opening; and
contacting the site marker positioned within the lumen of the delivery tube, thereby guiding the site marker out of the distal opening of the lumen of the delivery tube through the aperture of the outer cannula and into a biopsy cavity.

16. The method of claim 15, further comprising the step of protruding at least a portion of the push rod from the aperture a predetermined distance when the site marker is discharged from the aperture.
